(19) United States
(12) Patent Application Publication
(10) Pub. No.: US 2024/0309406 A1
(43) Pub. Date: Sep. 19, 2024

(54) PROCESS FOR PREPARING RASPBERRY KETONE

(71) Applicant: SPECIALTY OPERATIONS FRANCE, Lyon (FR)

(72) Inventors: Valéry Dambrin, Irigny (FR); Mirjana Gelo-Pujic, Sérézin-Du-Rhône (FR)

(73) Assignee: SPECIALTY OPERATIONS FRANCE, Lyon (FR)

(21) Appl. No.: 18/257,456

(22) PCT Filed: Dec. 14, 2021
(86) PCT No.: PCT/EP2021/085636

§ 371 Date: Jun. 14, 2023

(30) Foreign Application Priority Data
Dec. 14, 2020 (FR) FR2013142

(51) Int. Cl. C12P 7/26; C12N 1/18; C12N 9/02

(52) U.S. Cl. CPC C12P 7/26; C12N 1/185; C12N 9/001; C12N 2511/00

(57) ABSTRACT

The present invention relates to a process for preparing natural raspberry ketone, comprising a step (a) of bioconversion of p-coumaric acid allowing the preparation of p-hydroxybenzaldehyde, a step (b) in which the p-hydroxybenzaldehyde obtained on conclusion of step (a) is condensed with acetone to allow the formation of p-hydroxybenzalacetone, a step (c) in which the p-hydroxybenzalacetone obtained on conclusion of step (b) is converted into raspberry ketone by bioconversion or biocatalysis.

### FIELD OF THE INVENTION

The present invention relates to a process for preparing natural raspberry ketone.

### PRIOR ART

Raspberry ketone, or 4-(4-hydroxyphenyl)-2-butanone, is the main aromatic compound in raspberries, but is also present in cranberries or blackberries.

Raspberry ketone is used in perfumery, cosmetics or in the agrifood industry to give a fruity odour.

This natural aromatic compound may be extracted from fruits at a rate of 1 to 4 mg per kilogram of raspberries. Given the very low abundance of this aromatic compound in the fruit, synthetic processes have been developed, notably:
by alkylation of phenol in the presence of butenone as described in FR1227595;
by condensation of phenol in the presence of 4-hydroxy-2-butanone, as described in DE 2145308, CN104355977 or CN104496778. 4-Hydroxy-2-butanone is prepared by condensation of acetone and formaldehyde;
by condensation of phenol in the presence of 2-acetyl-2-hydroxymethylethyl acetate, as described in FR 2221433. The compound 2-acetyl-2-hydroxymethylethyl acetate is prepared from formaldehyde, and ethyl acetoacetate;
by condensation of phenol with 1,3-dichloro-2-butene, as described in JP01242549; or
by demethylation, in the presence of hydrobromic acid, of anisylacetone as described in CN104193607.

To the inventors' knowledge, only synthetic processes exist in an attempt to compensate for the low abundance of natural raspberry ketone. However, synthetic flavours are less appreciated by consumers than flavours of natural origin.

The present invention is directed towards manufacturing natural raspberry ketone via a novel route with good yields and high specificity.

### BRIEF DESCRIPTION

A first subject of the present invention relates to a process for preparing natural raspberry ketone, comprising:
a step (a) of bioconversion of p-coumaric acid allowing the preparation of p-hydroxybenzaldehyde,
a step (b) in which the p-hydroxybenzaldehyde obtained on conclusion of step (a) is condensed with acetone to allow the formation of p-hydroxybenzalacetone,
a step (c) in which the p-hydroxybenzalacetone obtained on conclusion of step (b) is converted into raspberry ketone by bioconversion or biocatalysis.

Another subject of the present invention relates to a natural raspberry ketone that may be obtained via the process of the present invention.

The present invention also relates to the use of natural raspberry ketone according to the present invention as a flavour or fragrance.

Finally, the present invention relates to a composition comprising natural raspberry ketone according to the present invention, preferably chosen from the group consisting of food products, beverages, cosmetic formulations, pharmaceutical formulations and fragrances.

### DETAILED DESCRIPTION

In the context of the present invention, and unless otherwise indicated, the expression “between . . . and . . . ” includes the limits. In the context of the present invention, unless otherwise indicated, the term “comprising . . . ” has the meaning of “consisting of . . . ”. Unless otherwise indicated, the percentages and ppm are percentages and ppm by mass.

In the context of the present invention, and unless otherwise indicated, the term “ppm” means “parts per million”. This unit represents a mass fraction: 1 ppm=1 mg/kg.

The natural raspberry ketone according to the invention, and obtained via the process of the invention, is a natural flavouring substance according to Article 9.2.c) of Regulation EC1334/2008. That is to say, a flavouring substance obtained via physical, enzymatic or microbiological processes from materials of plant, animal or microbiological origin taken as they are or after they have been processed for human consumption via one or more of the conventional processes for preparing foodstuffs. A natural flavouring substance corresponds to a substance which is naturally present and has been identified in nature.

In the context of the present invention, and unless otherwise indicated, the term “fermentation” refers to a microbiological process involving a microorganism. In general, the reaction is performed in a fermenter in a microbial culture medium.

In the context of the present invention, the term “fermentation” refers to a process comprising a growth step in which a microorganism is placed in a medium favouring its growth, followed by a bioconversion step in which said microorganism is placed in the presence of the substrate to be transformed; preferably, the microorganism is a bacterium or a yeast.

In the context of the present invention, and unless otherwise indicated, the term “biocatalysis” refers to a process step in which a substrate is transformed via enzymatic catalysis.

A first subject of the present invention relates to a process for preparing natural raspberry ketone, comprising:
a step (a) of bioconversion of p-coumaric acid allowing the preparation of p-hydroxybenzaldehyde,
a step (b) in which the p-hydroxybenzaldehyde obtained on conclusion of step (a) is condensed with acetone to allow the formation of p-hydroxybenzalacetone,
a step (c) in which the p-hydroxybenzalacetone obtained on conclusion of step (b) is converted into raspberry ketone via bioconversion or biocatalysis.

### Step (a):

The process for preparing raspberry ketone comprises a step (a) of bioconversion of p-coumaric acid to allow the preparation of p-hydroxybenzaldehyde according to the following scheme:

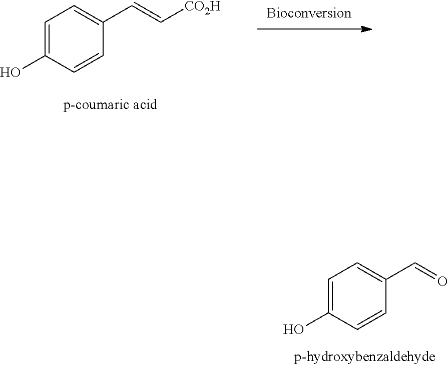 [SMILES: O=C(O)/C=C/c1ccc(O)cc1 ; O=Cc1ccc(O)cc1]

Step (a) is a bioconversion step: preferably, step (a) is a microbiological process. Generally, the bioconversion reaction is performed by fermentation in the presence of a microorganism. Preferably, the microorganism is chosen from bacteria belonging to the order Actinomycetales, preferably belonging to the family Streptomycetacae, Pseudonocardiacae, very preferentially Streptomyces setonii, Amycolatopsis sp. Streptomyces psammoticus. Preferably, the bioconversion reaction is performed in the presence of a strain available under the number ATCC39116, DSMZ 9991, DSMZ 9992, CCTCC 2015329 or IMI 390106.

Independently of the raspberry ketone preparation process, the bacterium used in step (a) is pre-cultured. The culturing of the bacterium is generally performed in an aqueous medium, in the presence of nutrient elements. Generally, the culture medium comprises a carbon source, preferably glucose, an organic or inorganic nitrogen source, inorganic salts and growth factors.

The concentration of the carbon source is generally between 5 and 50 g·L⁻¹, preferably between 20 and 34 g·L⁻¹. The nitrogen source, such as a yeast extract, and the growth factors are generally added at a concentration of between 2 and 20 g·L⁻¹, preferably between 5 and 10 g·L⁻¹. In addition, magnesium ions, such as magnesium sulfate, can be added in a concentration of between 0.1 and 5 g·L⁻¹, preferably between 0.5 and 1 g·L⁻¹. The temperature is generally between 25 and 50° C., preferably between 28° C. and 45° C. The pH of the culture is between 7 and 9.

The culture period generally lasts between 5 and 40 hours. The culture period generally lasts until the carbon source, usually glucose, is almost completely consumed, preferably such that the carbon source concentration is less than or equal to 1 g·L⁻¹.

The pH of the culture medium is then adjusted. Preferably, the pH is adjusted to a value greater than or equal to 8. Preferably, the pH is less than or equal to 11, preferentially less than or equal to 10.

The p-coumaric acid is then added to the culture medium obtained during this preculturing step. The concentration of p-coumaric acid in the culture medium is generally between 5 g·L⁻¹ and 50 g·L⁻¹. The addition of the p-coumaric acid may be performed in one go. The p-coumaric acid may also be added in several portions. The p-coumaric acid may be added alone or as a solution in an aqueous medium, preferably at a concentration of between 5 and 40 g·L⁻¹, preferably between 15 and 30 g·L⁻¹. The temperature of step (a) is generally between 25 and 50° C., preferably between 28° C. and 45° C.

The incubation time in the fermenter is generally between 5 and 50 hours, preferably between 15 and 25 hours. The pH in the fermenter is generally between 7 and 9. The p-coumaric acid is consumed and p-hydroxybenzaldehyde is obtained in the medium.

Other reaction by-products may also be obtained in the fermentation medium, such as p-hydroxybenzoic acid, or p-hydroxybenzyl alcohol.

At the end of the bioconversion step (a), the p-hydroxybenzaldehyde is recovered after removal from the biomass, via any method known to those skilled in the art. The p-hydroxybenzaldehyde may be used without further purification in step (b) or may be purified via any method known to those skilled in the art before use in step (b).

According to a particular embodiment, step (b) may be performed without intermediate purification; the p-hydroxybenzaldehyde obtained on conclusion of step (a) may be directly subjected to the conditions of step (b). Surprisingly, the impurities or by-products from step (a) have little or no impact on the conversion results of step (b).

Alternatively, step (b) may be performed in the presence of the biomass from step (a).

### Step (b):

The process for preparing raspberry ketone comprises a step (b) of condensation of p-hydroxybenzaldehyde with acetone according to the following scheme:

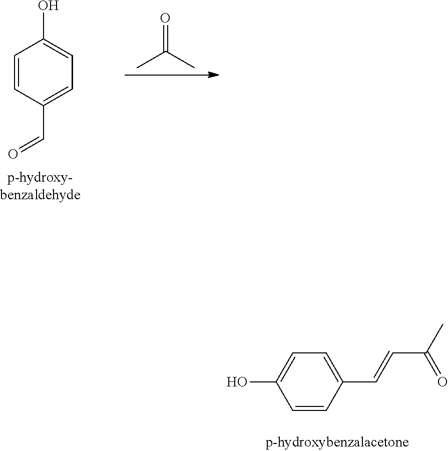

According to one aspect, the condensation step (b) is performed in the presence of a biocatalyst, preferably albumin, and more particularly in the presence of bovine serum albumin (BSA). Preferably, the reaction is performed in acetone. Generally, acetone is used in excess relative to the amount of p-hydroxybenzaldehyde. The reaction may be performed in the presence of another solvent chosen from water, a buffered aqueous medium, preferably at pH 8, ethanol, n-octane or ethyl acetate, preferably chosen from water, a buffered aqueous medium, preferably at pH 8, ethanol or ethyl acetate. According to an advantageous aspect, the solvent is chosen from solvents that are compatible for cosmetic or food applications.

The reaction is generally performed in the presence of a base, preferably chosen from imidazole, proline, L-histidine or guanidine hydrochloride.

The temperature of step (b) is generally between 30° C. and 40° C. Generally, step (b) is performed at atmospheric pressure. Generally, step (b) is performed with stirring, preferably at a speed of between 100 and 500 rpm, preferably between 150 and 300 rpm and very preferentially between 180 and 200 rpm. The incubation time of step (b) is generally between 24 and 96 hours. According to another aspect, step (b) may be performed in the presence of an amino acid, preferably an amino acid of the L series, preferably chosen from proline, azetidine-2-carboxylic acid, piperidine-2-carboxylic acid, 4-hydroxypyrrolidine-2-carboxylic acid, pyrrolidine-2-carboxamide, thiazolidine-4-carboxylic acid and 4-acetoxypyrrolidine-2-carboxylic acid. The amount of amino acid is generally between 15% by volume and 40% by volume. The solvent is generally a mixture of DMSO and acetone, or ethanol and water. According to an advantageous aspect, the solvent is chosen from solvents that are compatible for cosmetic or food applications.

These conditions are notably described in J. Am. Chem. Soc. 2000, 122 (10), 2395. However, contrary to what is described in said document, the reaction allows the predominant formation of the α,β-unsaturated ketone.

The p-hydroxybenzalacetone may be used without further purification in step (c) or may be purified via any method known to those skilled in the art before use in step (c).

According to a particular embodiment, step (c) may be performed without intermediate purification; the p-hydroxybenzalacetone obtained on conclusion of step (b) may be directly subjected to the conditions of step (c). Surprisingly, the impurities or by-products from step (b) and/or (a) have little or no impact on the conversion results of step (c).

### Step (c):

The process for preparing raspberry ketone comprises a step (c) in which p-hydroxybenzalacetone is hydrogenated according to the following scheme:

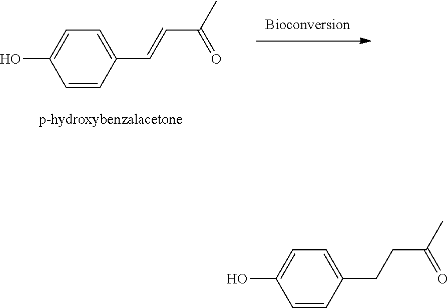

According to one aspect, step (c) is performed by biocatalysis.

According to one aspect, step (c) is performed in the presence of at least one enzyme; preferably, the enzyme is an ene-reductase or enone-reductase. Step (c) may also be performed in the presence of yeast, notably baker's yeast. Generally, step (c) is performed under reaction conditions that are suitable for the conversion of p-hydroxybenzalacetone to raspberry ketone. Generally, step (c) may be performed in the presence of a cofactor. A means of regenerating the cofactor may be used in combination with said cofactor. By way of illustration, the reduction in step (c) may be performed by generating NADP+ from NADPH, and thus any system capable of regenerating NADPH may be used.

Examples of systems that are capable of regenerating the cofactor that may be employed are glucose and glucose dehydrogenase, formate and formate dehydrogenase, glucose-6-phosphate and glucose-6-phosphate dehydrogenase, a secondary alcohol and ketone dehydrogenase, phosphite and phosphite dehydrogenase, molecular hydrogen and hydrogenase, flavin adenine dinucleotide FAD/FADH₂. These systems may be used with NADP+/NADPH or NAD+/NADH as cofactor.

According to another aspect, step (c) is performed by fermentation in the presence of a microorganism. The microorganism may be chosen from bacteria belonging to the order Actinomycetales, preferably belonging to the family Streptomycetacae, Pseudonocardiacae, very preferentially Streptomyces setonii, Amycolatopsis sp. Streptomyces psammoticus. Preferably, the bioconversion reaction is performed in the presence of a strain available under the number ATCC39116, DSMZ 9991, DSMZ 9992, CCTCC 2015329 or IMI 390106. The microorganism may also be chosen from yeasts belonging to the Saccharomyces or Candida group, preferably chosen from Saccharomyces cerevisiae and Candida lipolytica. Preferably, the reaction is performed in the presence of a strain available under the number ATCC7754 or ATCC 8661. The microorganism used in step (c) allows the carbon-carbon double bond to be selectively reduced without reducing the carbon-oxygen double bond.

Step (c) is generally performed in a solvent, preferably chosen from the group consisting of water, organic solvents and ionic liquids. Preferably, the organic solvents are chosen from the group consisting of ethyl acetate, butyl acetate, 1-octanol, heptane, octane, methyl-t-butyl ether (MTBE), ethanol and DMSO. According to an advantageous aspect, the solvent is chosen from solvents that are compatible for cosmetic or food applications. According to a particular aspect, the solvent may be an aqueous solvent comprising a mixture of water and another solvent. The aqueous solvent may be buffered or unbuffered. Generally, step (c) is performed at a pH of less than or equal to 10, preferably less than or equal to 9, more preferably less than or equal to 8. Generally, step (c) is performed at a pH of greater than or equal to 5, preferably greater than or equal to 6, more preferably greater than or equal to 7. During the reaction, the pH may be varied; it is possible to maintain the pH at a chosen value by adding a base or an acid. The pH may also be controlled with a buffer solution.

The order in which the reagents are added is not particularly critical. The reagents may be added together or separately in the chosen solvent. By way of illustration, the cofactor regeneration system, cofactor, ene-reductase or enone-reductase may be added first to the solvent.

Step (c) is generally performed at a temperature of between 15° C. and 75° C., preferably between 20° C. and 55° C., even more preferentially between 20° C. and 45° C. The reaction may also be performed at ambient temperature. The ambient temperature is generally between 19° C. and 26° C.

In general, step (c) is performed with stirring, preferably at a speed of between 100 and 500 rpm, preferably between 150 and 300 rpm and very preferentially between 180 and 200 rpm. The incubation time of step (c) is generally between 24 and 96 hours.

According to a particular aspect, the enzyme used for step (c) may be an enzyme such as the enzyme described in bioRxiv 202341; doi: https://doi.org/10.1101/202341.

According to a particular aspect, the enzyme used for step (c) may be an enzyme as described in WO 2010/075574.

Advantageously, the enzyme or microorganism used in step (c) is capable of specifically reducing the carbon-carbon double bond to obtain the formation of the ketone. Advantageously, the enzyme used in step (c) makes it possible to obtain raspberry ketone in a predominant manner relative to frambinol. The derivative frambinol corresponds to the following structure, in which the ketone function is reduced to an alcohol function.

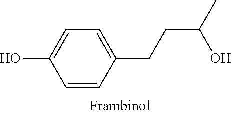

It is well known to those skilled in the art that the organoleptic properties of a flavouring substance may depend on the presence and amount of certain impurities. This is why the manufacturing process is essential for the taste of the final compound. Advantageously, the raspberry ketone of the present invention was found to have satisfactory organoleptic properties. It is noted that the organoleptic profile of the raspberry ketone of the present invention is equivalent to the organoleptic profile of raspberry ketone extracted from fruits.

Advantageously, the process of the present invention is capable of specifically producing natural raspberry ketone in good yields.

According to another aspect, the present invention covers the use of the raspberry ketone according to the present invention or the raspberry ketone obtained according to the process of the invention as a flavour or fragrance.

Finally, the present invention also covers a composition comprising raspberry ketone according to the invention, preferably chosen from the group consisting of food products, beverages, cosmetic formulations, pharmaceutical formulations and fragrances.

### EXAMPLES

### Example 1: Preparation of p-hydroxybenzaldehyde

### 1. Preculturing and Culturing

A preculture and culture medium suitable for ATCC39116, comprising KH₂PO₄, Na₂HPO₄·12H₂O, MgSO₄·7H₂O, yeast extract, glucose and antifoam is prepared. The preculturing is performed at 170 rpm and 37° C. The culturing is performed at 37° C. with stirring.

### 2. Bioconversion

The pH of the medium is adjusted to 8.4 and a coumaric acid solution is introduced so as to obtain a final concentration of between 5 and 50 g/L. The reaction medium is maintained at 37° C. and 170 rpm for 24 h. After 24 h of bioconversion, the biomass is removed by centrifugation, and the supernatant is filtered off and analysed by HPLC. 4-Hydroxybenzaldehyde is obtained, in a yield of between 60% and 99%.

### Example 2: Preparation of Benzalacetone

p-Hydroxybenzaldehyde, acetone, a solvent, a base and BSA are mixed together. The reaction medium is incubated at a temperature of between 30° C. and 40° C. and at 200 rpm for 24 to 96 hours. The reaction is monitored by HPLC. Benzalacetone is obtained with a degree of conversion of greater than or equal to 90%.

### Example 3a: Preparation of Raspberry Ketone by Biocatalysis

The benzalacetone obtained in Example 2, a solvent, GDH, glucose, NADP+, KH₂PO₄ pH 7 and an ene-reductase are mixed together. The mixture is incubated at 30° C. with stirring for 24 h. The media are analysed by HPLC.

Raspberry ketone is obtained. Degree of conversion of the substrate=100%

Selectivity: 100% reduction of the C═C double bond

### Example 3b: Preparation of Raspberry Ketone by Bioconversion

### 1. Preculturing

A preculture medium suitable for microorganisms 1 and 2 indicated in the table below, comprising glucose, peptone and malt extract, is prepared. Microorganisms 1 to 3 are then added directly to the preculture medium. Culturing is performed at 30° C. with stirring (200 rpm) for 24 hours.

A preculture and culture medium suitable for microorganism number 3 is prepared in accordance with Example 1 above.

| Reference | Strain |
|---|---|
| 1 | Candida lipolytica ATCC 8661 |
| 2 | Saccharomyces cerevisiae ATCC 7754 |
| 3 | Amycolatopsis sp. ATCC 39116 |

### 2. Bioconversion

The benzalacetone obtained according to Example 2 is mixed into each medium obtained after the growth period as described above. The mixture is incubated at 30° C. with stirring (200 rpm) for 48 hours (references 1 to 2) or 72 hours (reference 3).

After 48 h or 72 h of bioconversion, the biomass is removed by centrifugation, and the supernatant is filtered off and analysed by HPLC. raspberry ketone is obtained.

1. A process for preparing natural raspberry ketone, comprising:
a step (a) of bioconversion of p-coumaric acid allowing for preparation of p-hydroxybenzaldehyde,
a step (b) in which the p-hydroxybenzaldehyde obtained upon conclusion of step (a) is condensed with acetone to allow for formation of p-hydroxybenzalacetone,
a step (c) in which the p-hydroxybenzalacetone obtained upon conclusion of step (b) is converted into raspberry ketone by bioconversion or biocatalysis.

2. The process according to claim 1, in which step (a) and/or step (c) is a microbiological process.

3. The process according to claim 1, in which the bioconversion reaction is performed by fermentation in the presence of a microorganism.

4. The process according to claim 1, in which the condensation step (b) is performed in the presence of a biocatalyst.

5. The process according to claim 1, in which step (b) is performed in the presence of an amino acid.

6. The process according to claim 1, in which step (c) is performed in the presence of an ene-reductase or an enone-reductase.

7. The process according to claim 1, in which step (c) is performed in the presence of a cofactor.

8. The process according to claim 1, in which step (c) is performed in the presence of a yeast belonging to the group Saccharomyces or Candida.

9. The process according to claim 1, in which the p-hydroxybenzaldehyde obtained on conclusion of step (a) is used without further purification in step (b).

10. The process according to claim 1, in which the p-hydroxybenzalacetone obtained on conclusion of step (b) is used without further purification in step (c).

11. The process according to claim 3, wherein the microorganism comprises one or more microorganisms belonging to the order of Actinomycetales, the family Streptomycetacae, Pseudonocardiacae, or any combination thereof.

12. The process according to claim 11, wherein the microorganism comprises Streptomyces setonii, Amycolatopsis sp. Streptomyces psammoticus, or both.

13. The process according to claim 11, wherein the microorganism comprises a strain available under the number ATCC39116, DSMZ 9991, DSMZ 9992, CCTCC 2015329 or IMI 390106.

14. The process according to claim 4, wherein the biocatalyst is an albumin.

15. The process according to claim 14, wherein the albumin is bovine serum albumin.

16. The process according to claim 8, wherein the yeast comprises Saccharomyces cerevisiae, Candida lipolytica, or both.
